(19) United States
(12) Patent Application Publication
(10) Pub. No.: US 2002/0150609 A1
(43) Pub. Date: Oct. 17, 2002

(54) Cosmetic sheet product

(76) Inventors: Yasuhiro Kono, Sagamihara-city (JP); Jayshree Seth, Woodbury, MN (US); Kenjiroh Kotoh, Yamato City (JP)

Correspondence Address: Office of Intellectual Property Counsel, 3M Innovative Properties Company, PO Box 33427, St.Paul, MN, 55133-3427, US

(21) Appl. No.: 09/780,257

(22) Filed: Feb. 9, 2001

(51) Int. Cl. A61K 7/50; B32B 27/12; C11D 1/00

(52) U.S. Cl. 424/443; 442/123; 510/130

(57) ABSTRACT

A package containing at least one soft fibrous wipe laminate; the wipe laminate comprising a nonwoven wipe layer formed from a nonwoven web comprised predominantly of thermoplastic polymer, copolymer or blend fibers; and a backing layer bonded to the wipe layer where the nonwoven fibrous web has bonded regions and unbonded regions the unbonded region forming the arcuate mounds.

### BACKGROUND OF THE INVENTION

This invention is related to discrete sheets for applying active or cleansing agents to the skin. More particularly, the invention is related to a disposable nonwoven sheet that is used to apply or dispense topically active agents or cleansers to the skin.

Nonwoven products are commonly used to carry and/or apply surface active agents onto the skin. U.S. Pat. No. 5,605,749 suggests that it is desirable for a polishing pad allegedly for personal or industrial use to have a combination of a compression resilience, such that the amount of release of an active agent applied by the sheet can be controlled by applying varying levels of hand pressure if needed and be re-absorbed when the pressure is reduced; thorough release of the absorbed active agent during use; high physical strength; abrasion resistance; and non-abrasive so that the pad does not abrade or damage the target surface. The wipe is formed of conjugate fibers having at least two crimps per inch. This provides a wipe with the requisite amount of loft and compression resistance to provide the desired properties. The pad described in U.S. Pat. No. 5,605,749 can be used for diverse applications including automotive or cosmetic uses. The proposed pad is fabricated from a nonwoven web that contains crimped conjugate fibers of spunbond fibers or staple fibers where the nonwoven is characterized as having autogenous interfiber bonds at the crossover contact points of its fibers throughout the web, and is impregnated with a topically applicable active agent. The crimped conjugate fibers have at least 2 crimps per extended inch (2.54 cm) as measured in accordance with ASTM D-3937-82. The nonwoven pad showed good absorbent capacity and delivery of active agent under pressure compared to a nonlofting nonwoven pad.

Various patents have generally proposed that nonwoven, woven or the like webs can be used to either apply active agents to the skin where the active agents can be impregnated into the web or wipe prior to use or be impregnated or applied to the wipe by the user. For example, U.S. Pat. No. 5,744,149 forms a medicated pad using a laminate of a paper layer with a synthetic nonwoven layer. U.S. Pat. No. 1,752,765 discloses a woven pad for applying powders. U.S. Pat. No. 967,688 describes a paper wipe impregnated with salicylic acid. U.S. Pat. No. 2,187,163 describes a cotton or felt pad impregnated with a deodorant composition.

U.S. Pat. Nos. 3,537,121 and 3,910,284 disclose a buffing pad that cleans or restores luster without scratching or abrading the target surface that is being cleaned or buffed. The buffing pad is fabricated from a synthetic fiber web that is bonded with an elastomeric binder. This is a relatively low loft pad. U.S. Pat. No. 4,775,582 to Abba et al. discloses a meltblown nonwoven wet wipe for personal care uses. This also is a low loft pad.

European Patent No. 750 062 proposed generally that the use of nonwoven webs with a basis weight of from 20 to 130 g/m² are useful as skin cleansing wipes. The wipes preferably have a specific coefficient of friction and are produced by hydroentanging, needlepunching or the like. The described wipes allegedly have improved softness and are less harsh on the skin compared to woven cloths or paper based pads and are allegedly stronger than cotton based nonwoven wipes.

It is desirable for cosmetic or skin applications to provide a wipe construction that has the functional properties of the wipe described in U.S. Pat. No. 5,605,749 with high absorption capacity for active agents, good releasability of absorbed active agents and a good combination of softness and strength, which wipe and can be formed from a broader range of nonwoven fabrics and fibers in its construction (e.g., not be limited to the use of conjugate fibers having at least two crimps per inch or nonwovens that are bonded at all fiber crossover points).

### SUMMARY OF THE INVENTION

The present invention is related to a soft nonwoven fibrous cosmetic wipe material for personal use applications. The nonwoven fibrous wipe material of the invention contains a nonwoven fibrous layer having a z-direction loft from the backing of at least 0.5 mm where the nonwoven fibrous layer material is bonded to a backing layer. The z-direction loft is formed by arcuate portions between areas of bonding of the nonwoven layer to the backing which arcuate portions comprise from 20 to 99 percent of the wipe cross-sectional wiping area. The wipe is further preferably provided with an agent for use with a user's skin or hair. The active agent is preferably impregnated into the wipe.

### BRIEF DESCRIPTION OF THE DRAWINGS

The present invention is further described in reference to accompanying drawings, where unlike reference numerals refer to like parts on several views, and wherein:

FIG. 1 is a perspective view of a first embodiment of sheet of wipes prepared according to the present invention.

FIG. 2 is a schematic view illustrating a method of forming the wipe material of the invention depicted in FIG. 1.

FIG. 3 is a schematic view of a second embodiment for producing the wipe material of FIG. 1.

FIG. 4 is a side view of a corrugating member which could be substituted for the corrugating members illustrated in FIG. 2 or FIG. 3.

FIG. 5 is a side view of a second corrugating member which could be substituted for the corrugating members illustrated in FIG. 2 or FIG. 3.

FIG. 6 is a perspective view of a wipe in a package.

FIG. 7 is a perspective view of a wipe in use.

### DETAILED DESCRIPTION OF THE INVENTION

The present invention provides a nonwoven wipe that is highly suitable for impregnating a large amount of topically applicable active agents or cleansing agents and releasing the impregnated active or cleansing agents on demand with low levels of hand pressure.

Fibers suitable for forming the nonwoven fibrous layer of the present invention nonwoven wipes can be produced from a wide variety of thermoplastic polymers that are known to form fibers. Suitable thermoplastic polymers are selected from polyolefins, polyamides, polyesters, copolymers containing acrylic monomers, and blends and copolymers thereof. Suitable polyolefins include polyethylene, e.g., linear low density polyethylene, high density polyethylene, low density polyethylene and medium density polyethylene; polypropylene, e.g., isotactic polypropylene, syndiotactic polypropylene, blends thereof and blends of isotactic polypropylene and atactic polypropylene; and polybutylene, e.g., poly(1-butene) and poly(2- butene); polypentene, e.g., poly-4-methylpentene-1 and poly(2-pentene); as well as blends and copolymers thereof. Suitable polyamides include nylon 6, nylon 6/6, nylon 10, nylon 4/6, nylon 10/10, nylon 12, nylon 6/12, nylon 12/12, and hydrophilic polyamide copolymers such as copolymers of caprolactam and an alkylene oxide, e.g., ethylene oxide, and copolymers of hexamethylene adipamide and an alkylene oxide, as well as blends and copolymers thereof. Suitable polyesters include polyethylene terephthalate, polybutylene terephthalate, polycyclohexylenedimethylene terephthalate, and blends and copolymers thereof. Acrylic copolymers include ethylene acrylic acid, ethylene methacrylic acid, ethylene methylacrylate, ethylene ethylacrylate, ethylene butylacrylate and blends thereof. Particularly suitable polymers are polyolefins, including polyethylene, e.g., linear low density polyethylene, low density polyethylene, medium density polyethylene, high density polyethylene and blends thereof; polypropylene; polybutylene; and copolymers as well as blends thereof.

The nonwoven wipe of the invention in general are oleophilic since most of the above described fiber-forming polymers are naturally oleophilic. Consequently, oil based active agents and emulsified active agents are readily absorbed and retained by the nonwoven wipe. When aqueous or hydrophilic active agents are desired to be impregnated in the nonwoven wipe, the fibers of the nonwoven web that forms the wipe may be hydrophilic fibers or hydrophilically modified. Hydrophilic fibers include natural or synthetic fibers such as cotton fibers, cellulosic fibers, rayon and the like. Cotton or other non-thermoplastic fibers, if used are preferably blended with thermoplastic fibers such that the nonwoven wipe has at least 50 percent thermoplastic fibers by weight, preferably 75 percent thermoplastic fibers. Further, any of a wide variety of surfactants, including ionic and nonionic surfactants, may be employed to hydrophilically modify the nonwoven web or fibers. Suitable surfactants may be internal modifiers, i.e., the modifying compounds are added to the polymer composition prior to spinning or forming fibers, or topical modifiers, i. e., the modifying compounds are topically applied during or subsequent to the formation of fibers or nonwoven webs. An exemplary internal modification process is disclosed in U.S. Pat. No. 4,578,414 to Sawyer et al. An exemplary topical modification process is disclosed in U.S. Pat. No. 5,057,361 to Sayovitz et al. Illustrative examples of suitable surfactants include silicone based surfactants, e.g., polyalkylene-oxide modified polydimethyl siloxane; fluoroaliphatic surfactants, e.g., perfluoroalkyl polyalkylene oxides; and other surfactants, e.g., actyl-phenoxypolyethyoxy ethanol nonionic surfactants, alkylaryl polyether alcohols, and polyethylene oxides. Commercially available surfactants suitable for the present invention include various poly(ethylene oxide) based surfactants available under the tradename Triton, e.g., grade X-102, from Rohm and Haas Crop; various polyethylene glycol based surfactants available under the tradename Emerest, e.g., grades 2620 and 2650, from Emery Industries; various polyalkylene oxide modified polydimethylsiloxane based surfactants available under the tradename Silwet, e.g., grade Y12488, from OSI Specialty Chemicals; and alkenyl succinamide surfactants available under the tradename Lubrizol, e.g., grade OS85870, from Lubrizol Crop.; and polyoxyalkylene modified fluoroaliphatic surfactants available from Minnesota Mining and Manufacturing Co. The amount of surfactants required and the hydrophilicity of modified fibers for each application will vary depending on the type of surfactant selected and the type of polymer used. In general, the surfactant may be added, topically or internally, in the range of from about 0.1 to about 5%, desirably from about 0.3 percent to about 4%, by weight based on the weight of the fiber or the nonwoven web.

In accordance with the present invention, a wide variety of topically applicable active agents can be impregnated into and used with the present nonwoven wipe, which include oil based active agents, e.g. mineral oil; emulsified active or cleansing agents, e.g., soaps, detergents, body lotions and emulsions; aqueous active agents, e.g., dermatological medicaments, germicidal solutions and bleaches; and others, e.g., alcohols, perfumes and dermatological cleansers, cosmetics, glitter, etc. By active agent, it is meant any agent that can be used on the skin or hair or can modify or provide a benefit to the skin or hair, including cleansing agents.

The active agents can be impregnated by the user or preimpregnated into the nonwoven wipe by any conventional techniques useful for impregnating or applying liquid or powders on or into a porous material, such as spraying, dipping, coating and printing. Optionally, once the nonwoven pad is impregnated with an active agent, the liquid content of any liquid containing active agent can be evaporated to provide a lower weight nonwoven pad that can be reactivated if needed by subsequently applying an appropriate solvent or water, or the nonwoven pad can be packaged as wet.

Preformed fibers can be formed into the nonwoven fibrous web by any suitable method such as carding, rando webbers, hydroentanging, and needlepunching. Alternatively, the nonwoven fibrous web can be directly formed from thermoplastic fiber forming polymers such as by spunbond or meltblown and like techniques that directly form nonwovens from a polymer melt. These nonwovens can be modified by blending in additional discrete fiber or particulates, coated or include suitable melt additives for the intended end use. Generally, the nonwoven fibrous web used to form the invention nonwoven wipe will be from 10 to 100 g/m², preferably 15 to 50 g/m² and comprise at least in part thermoplastic fibers suitable for bonding, preferably at least 10 percent bondable thermoplastic fibers, most preferably 20 to 100 percent bondable thermoplastic fibers.

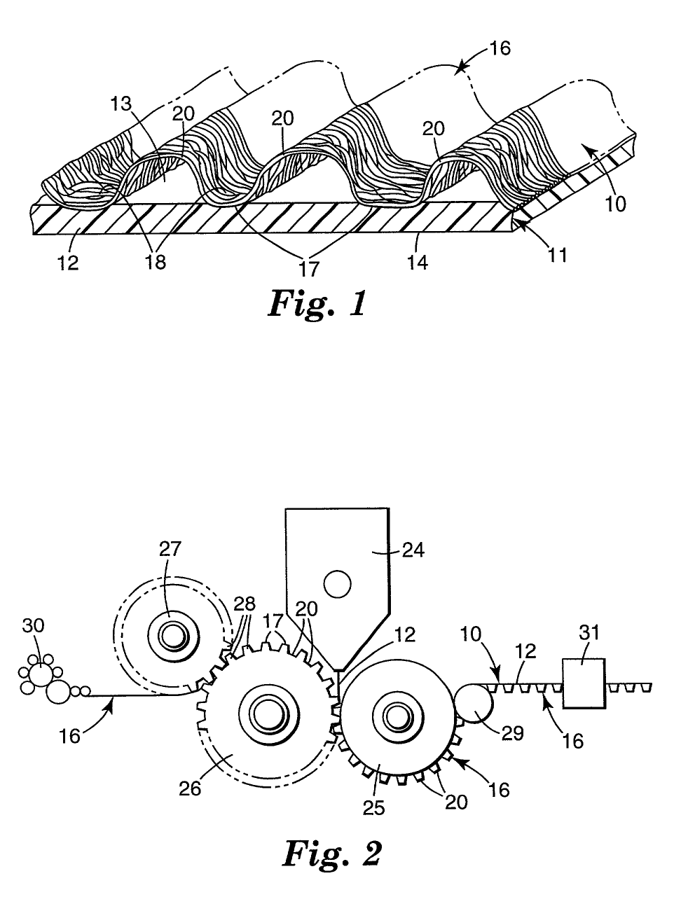

FIG. 1 illustrates a first embodiment of a nonwoven material useful in the present invention, generally designated by the reference numeral 10 which nonwoven laminate material 10 is cut into pieces to form individual personal use wipes. Generally the nonwoven laminate material 10 has a backing 11 comprising a thermoplastic film or nonwoven backing layer 12. The backing layer 12 in this embodiment is preferably a nonwoven, a film or parallel filament or strands, or laminates thereof, with front and rear surfaces 13 and 14. The nonwoven web 16 generally has non-deformed anchor portions 17 autogeneously bonded to the backing layer 12. The bonding locations 18 in FIG. 1 are along the front surface 13 with arcuate portions 20 of the nonwoven web 16 projecting from the front surface 13 of the backing layer 12 between the bonding locations 18. As shown in FIG. 1 the bonding locations can be continuous rows extending transversely across the nonwoven laminate material 10. However the bonding locations can be arranged in any pattern including, for example, intermittent lines, hexagonal cells, diamond cells, square cells, random point bonds, patterned point bonds, crosshatched lines, or any other regular or irregular geometric pattern.

FIG. 2 schematically illustrates a method and equipment for forming the wipe material 10 shown in FIG. 1. The method illustrated in FIG. 2 generally comprises forming a nonwoven fiber web 16 so that it has arcuate portions 20 projecting in the same direction from spaced generally parallel anchor portions 17 of nonwoven web 16, and bonding the spaced generally parallel anchor portions 17 of the nonwoven web 16 to the backing layer 12. This method is performed in the FIG. 2 method by providing first and second corrugating members or rollers, 26 and 27 each having an axis and including a plurality of circumferentially spaced generally axially extending ridges 28 around and defining its periphery, with spaces between the ridges 28 adapted to receive portions of the ridges 28 of the other corrugating member, 26 or 27, in meshing relationship with the nonwoven web 16 between the meshed ridges 28. The corrugating members 26 and 27 are mounted in axially parallel relationship with portions of the ridges 28 meshing generally in the manner of gear teeth; at least one of the corrugating members, 26 or 27, is rotated; and the nonwoven web of 16 is fed between the meshed portions of the ridges 28 of the corrugating members 26 and 27 to generally corrugate the sheet of fibers 16. The corrugated nonwoven web 16 is retained along the periphery of the first corrugating member 26 after it has moved past the meshed portions of the ridges 28. In the FIG. 2 method a thermoplastic backing layer 12 of a film or a plurality of closely spaced filaments is formed and bonded to the anchor portions 17 of the sheet of fibers 16 on the end surfaces of the ridges 28 on the first corrugating member 26 by extruding or coextruding the thermoplastic backing layer 12 in a molten state from a die 24 into a nip between the anchor portions 17 of the nonwoven 16 on the periphery of the first corrugating member 26 and a cooling roll 25. This embeds the fibers of the nonwoven web into the film or filament backing layer. After cooling by the cooling roll 25 in the nip the sheet of loop material 10 is separated from the first corrugating member 26 and carried partially around the cooling roll 25 and through a nip between the cooling roller 25 and a pinch roller 29 to complete cooling and solidification of the backing layer 12.

An alternative to extruding a film or a plurality of closely spaced filaments is supplying a preformed film or nonwoven backing layer, into the nip formed between the first corrugating member 26 and a roll 25. The ridges on the corrugating member 26 and/or the roll 25 are heated so as to thermally bond the backing to the sheet of nonwoven fibers. Alternatively, adhesive could be used to bond the backing layer onto the nonwoven web 16.

The nonwoven fibrous web can be formed from discrete fibers using, e.g., a carding machine 30, which nonwoven web of randomly oriented fibers 16 has enough integrity to be fed from the, carding machine 30 into the nip between the corrugating members 26 and 27 (if needed, a conveyer (not shown) could be provided to help support and guide the nonwoven web 16 between the carding machine 30 and the corrugating members 26 and 27). When such a nonwoven web 16 is used, preferably the first corrugating member 26 has a rough finish (e.g., formed by sand blasting), the second corrugating member 27 has a smooth polished finish, and the first corrugating member 26 is heated to a temperature slightly above the temperature of the second corrugating member 26 so that the nonwoven web 16 will preferentially stay along the surface of the first corrugating member 26 and be carried to the nip between the first corrugating member and the roller 25 after passing through the nip between the corrugating members 26 and 27. Alternatively, a vacuum could be used to help hold the nonwoven fibrous web 16 onto the structure of the first corrugating member 26.

Optionally, the backing 11 of the nonwoven laminate 10 can be printed or impregnated with an active agent on its surface opposite the nonwoven 16 through the use of a printer or coater 31, either in the production line as illustrated, or as a separate operation. For example, a printer or coater 31 could be used to print on or impregnate a pattern of ink or active agent on the nonwoven 16 either in the production line as illustrated or as a separate operation.

Corrugating members 26 and 27, as shown in FIG. 2, adapted to have a nonwoven fibrous web 16 fed into them, can have ridges 28 oriented generally in the range of 0 to 45 degrees with respect to its axes, but preferably have its ridges 28 oriented at 0 degrees with respect to (or parallel to) its axes which simplifies making of the corrugating members 26 and 27.

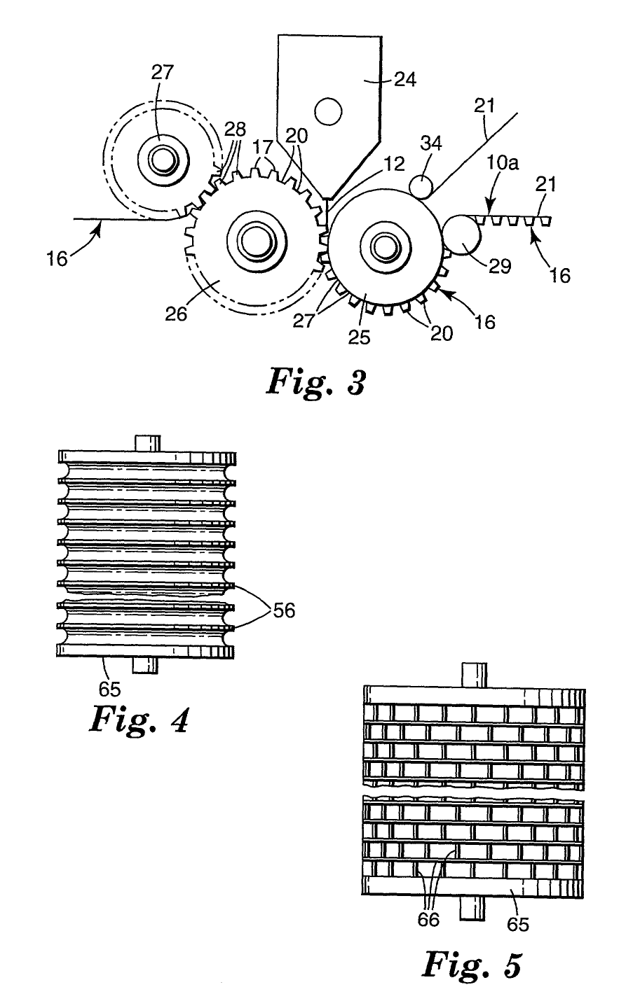

FIG. 3 schematically illustrates a second embodiment for forming nonwoven materials 10a, as shown in FIG. 2, which method is generally the same and uses much of the same equipment as is illustrated in FIG. 2 (with similar portions of that equipment having the same reference numerals), except for the addition of means including a pinch roller 34 for feeding the sheet of backing material 21 into the nip between the first corrugating roller 26 and the roller 25 along the surface of the roller 25 which results in the extruded molten thermoplastic backing or bonding layer 12 from the die 24 being deposited between the nonwoven 16 and of backing material 21. The nonwoven laminate 10a is then separated from the first corrugating member 26 and carried partially around the cooling roll 25 with its backing 11a against the cooling roll 25 to complete cooling and solidification of its thermoplastic backing layer 12.

The cooling roll 25 in the embodiments shown in FIGS. 2-3, can be water cooled and have a chrome plated periphery. Alternatively, the cooling roll 25 may have an outer rubber layer defining its surface which may be preferred for forming the nonwoven material 10a if the backing material 22 is of a material (e.g., paper) that tends to restrict heat transfer into the cooling roll 25. If roll 25 is a heated roll this could be by means of an oil or water heated roll or an induction roll.

The backing material 21 incorporated in the backing 11 could be a woven, knitted, needle punched, nonwoven (e.g., spunbond, hydroentangled web, carded web or the like) or other solid or porous layer of intertwined fibers, or could be a continuous polymeric film or continuous parallel filaments. Backing material 12, 21 or 11 may be single or multiple layer(s). If the backing is a series of closely spaced parallel filaments, the filaments are generally 0.5 to 10 mm apart, preferably 0.8 to 5 mm. If the backing is a porous material it could be preimpregnated or impregnated with an active agent, or if the backing is a nonporous material it could be coated with an active agent. The backing is at least in part preferably formed of a thermoplastic material for bondability to the nonwoven layer. A nonwoven backing could also include or be formed of absorbent fibers such as cellulosic fibers or other fibrous materials as described for the nonwoven wipe layer. A film backing could be a porous or nonporous film. With porous films, these could be used to remove skin oil.

Preferably for an extrusion bonded or thermally bonded method using corrugating rolls 26 and 27 and a nip roll 25, the drives for the corrugating members 26 and 27 and for the roller 25 can be rotated at a surface speed that is the same as or different than, the surface speed of the first corrugating member 26. When the roller 25 and the first corrugating member 26 are rotated so that they have the same surface speed, the nonwoven 16 will have about the same shape along the backing 11 as it had along the periphery of the first corrugating member 26 as is illustrated in FIGS. 2 and 3. When the roller 25 and the first corrugating member 26 are rotated so that the roller 25 has a surface speed that is slower than the surface speed of the first corrugating member 26, (e.g., one quarter or one half) the anchor portions 17 of the nonwoven 16 will be moved closer together in the backing layer 12 at the nip between the roller 25 and the first corrugating member 26, resulting in greater density of the arcuate portions 20 along the backing 11 than when the cooling roller 25 and the first corrugating member 26 are rotated so that they have the same surface speed.

FIGS. 4 and 5 illustrate two different corrugating members. One or a pair of cylindrical heated corrugating members 65 could be substituted for the corrugating member 26 and 27 to form a nonwoven wipe using generally the methods described above with reference to FIGS. 2 and 3. The corrugating member 65 and its mating corrugating member, if provided, each have an axis and includes a plurality of ridges 56 or 66. The ridges 66 or 56 on each corrugating member defining spaces between the ridges 56 or 66, which spaces can be adapted to receive a portion of the ridges of another corrugating member in meshing relationship in the manner of a pair of gears. If desired, the ridges on a first corrugating member could be arranged in any suitable pattern including forming words, numbers or symbols, for example, to form a trademark on the nonwoven material.

The arcuate portions of the nonwoven web between adjacent bonding locations provide the z-direction loft and have a generally uniform maximum height from the backing layer of less than about 10 mm and preferably 1 to 5 mm. The height of the arcuate portions of the nonwoven fibrous is at least one third, and preferably one half to one and one half times the distance between adjacent bonding locations. The arcuate portions generally comprise at least 20 percent of the cross-section of the wipe area preferably 50 to 95%. These arcuate portions provide for the wipe to have increased foaming action and skin contact when applying an active agent as well as provide high absorption capacity and releasability of absorbed active agents. The increased void volume also allows for storage of detritus from the skin.

The majority of the individual fibers forming the nonwoven fibrous web are preferably on average 1 to 70 μm in diameter. Preferably 40 to 70 μm where abrasiveness is desired and 1 to 30 μm where a soft product is desired. Preferably at least in part some of the fibers are compression resistant fibers which are generally fibers of 30 to 70, for polypropylene fibers, however this diameter depends on the nature of the material forming the fiber, these fibers could be blends with the soft fibers as desired. Larger diameter fibers are preferred where an abrasive action is desired, such as for skin exfoliation. Smaller diameter fibers are desired for more sensitive skin or removal of makeup or the like. The nonwoven fibrous web material, without the backing, has a basis weight in the range of 10 to 100 g/m² (and preferably in the range of 15 to 50 g/m²) measured along the first surface 13. The backing layer generally has a basis weight of from 15 to 150 g/m², preferably from 20 to 50. The total nonwoven laminate 10 has a basis weight of from 30 to 300 g/m², preferably 40 to 100.

If the nonwoven is a nonwoven fibrous web material provided by carding Rando webs, airlaid webs, spun-lace webs, spun-bond webs, or the like, the nonwoven fibrous material is preferably not prebonded or consolidated to maximize the open area between the fibers. However, in order to allow preformed webs to be handled, it is necessary on occasion to provide suitable point bonding and the like which should be at a level only sufficient to provide integrity to unwind the preformed web from a roll and into the forming process for creating the invention nonwoven fibrous laminate material.

Generally, the nonbonded portions of the nonwoven fibrous web is from 99.5 to 50 percent providing bonding areas over from 50 to 0.5 percent of the cross sectional surface area the nonwoven fibrous web, preferably the overall bonded area of the nonwoven is from 20 to 2 percent. The bonded areas include those areas of the sheet of fibers bonded to the backing layer as well as any prebonded or consolidated areas provided to improve web integrity. The specific bonding portions or areas bonded to the backing layer generally can be any width; however, preferably are from 0.01 to 0.2 centimeters in its narrowest width dimension. Adjacent bonding portions are generally on average spaced from 0.1 to 2.0 cm, and preferably 0.2 to 1.0 cm, apart. When the bonded portions are in the form of point bonds, the points are generally of substantially circular shape providing circular bonds preferably formed either by extrusion bonding or thermal bonding. Other shapes in the bonded and unbonded portions are possible, providing unbonded mounds or arcuate portions which are circular, triangular, hexagonal, or irregular in shape. The basis weight of a nonwoven layer is substantially increased when corrugated.

In order to maintain the desirable softness of the nonwoven wipe material, the backing layer or layers generally has a thickness from 10 to 300 microns, preferably from 20 to 100 microns providing a soft nonwoven fibrous loop material laminate having an overall circular bend stiffness of less than 9N, preferably less than 7N, and most preferably from 6N to 1N, while also providing a wipe material having sufficient tensile strength in order to be reliably used in continuous manufacturing techniques requiring a dimensionally stable material.

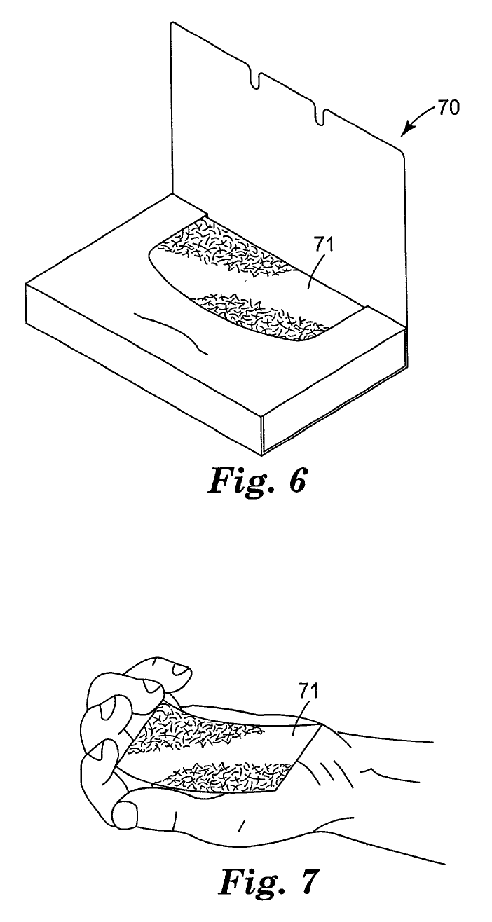

The individual discrete wipes 71 can be of any suitable size, however, generally for most applications the wipes would have an overall surface area of from 10 to 100 cm². preferably from 20 to 50 cm² suitable for easy handling as shown in FIG. 7. As such, the wipes would be of a size suitable for insertion in a package 70 as shown in FIG. 6, which could easily be placed in the user's purse or pocket. The material forming the dispensable containers is generally not of importance and can be formed of suitable papers, plastics, paper film laminates and the like. The shape of the wipes 71 is generally rectangular; however, other suitable shapes such as oval, circular or the like can be used. Generally, the discrete wipes would be provided in a package containing multiple wipes, e.g., more than 2, preferably at least 10.

### Test Methods

Capacity, Delivery and Efficiency

To measure the capacity and delivery characteristics of the wipe of the present invention to retain and release fluids, the following procedure was used. A dry 5 cm by 5 cm sample of wipe material was weighed to the nearest tenth of a gram (W1), followed by soaking the sample in water or mineral oil for 1 minute. The sample was then allowed to drip by gravity for 1 minute to remove any excess liquid and weighed again (W2). To measure the ability of the material to release liquid, a 2000 gram weight was placed onto the test sample for 1 minute, removed, and then weighed again (W3). The Absorbent Capacity represents the amount of liquid that the web can hold per unit weight of dry web. Delivery is the amount of liquid that the wet web can release under a load per unit weight of dry web. Efficiency is a measure of how much liquid the wet web can release expressed as a percent of the amount of liquid the web can hold. The results in Table 1 below are reported as:

Absorbent Capacity=(W2−W1)/W1

Delivery=(W2−W3)/W1

Efficiency=(Delivery/Absorbent Capacity)×100

Foam Height

To measure the ability of the wipes of the present invention to form a foam with a cleansing solution, the following procedure was used. A 2 mL pipette was to place 0.5 mL of liquid soap (Foaming Face Wash available from Olay) onto a 5 cm by 5 cm sample of web. 1 mL of water was then placed onto the surface of the web to wet the sample. The sample was then placed onto a flat metal plate and with gentle hand and finger pressure rubbed in a rotary manner against the plate for 20 revolutions at about 2.5 revolutions per second to generate a foam. The sample was then removed from the plate. The edge of the sample was then held and a wooden tongue depressor (held on edge) was drawn across the surface of the sample to remove the foam. The height of the foam on the depressor was marked with a pencil and then measured with a ruler. An average of 4 replicates was used and is reported in mm in Table 2 below.

### Comparative Examples

C1: 25 gram per square meter flat spunlaced nonwoven made from a blend of 60 percent cotton fibers and 40 percent polyester fibers available as “Escotto” from Unitika Corp. of Japan.

C2: 60 gram per square meter flat spunlaced nonwoven made from a blend of 60 percent rayon fibers and 40 percent polyester fibers available from Veratec Corp. of Walpole, Mass.

C3: 15 gram per square meter flat spunbond polypropylene nonwoven available from Avgol Corp. of Holon, Israel.

C4: 15 gram per square meter flat spunbond polypropylene nonwoven available from Freudenberg Nonwovens of Lowell, Mass.

C5: 30 gram per square meter flat spunbond type polypropylene nonwoven available as RFX from BP Amoco of Chicago, Ill.

C6: CelCels commercially available cosmetic applicator from Asahi of Japan.

C7: Silcot commercially available cosmetic applicator from Unicharm of Japan.

### EXAMPLES

### Example 1

A wipe of material was prepared using the method illustrated and described in U.S. Pat. No. 5,643,397 by feeding the nonwoven web C1 above into the nip between a first and second intermeshing corrugating rollers which were machined with axially parallel ridges spaced such that there were approximately 4 ridges per centimeter with a groove between each ridge. Each ridge was machined to have a flat top-surface having a width of about 0.7 mm. The corrugated sheet of nonwoven was shaped such that there were arcuate portions and anchor portions along the length of the nonwoven, each arcuate portion being about 0.33 cm high and about 0.33 centimeter long along the length of the nonwoven, and each anchor portion being about 0.07 centimeter wide. The first corrugating roller was heated to 93° C., whereas the second corrugating roller was heated to 149° C. A blend of polypropylene impact copolymer (75%) commercially designated “7C50” available from the Union Carbide Corp. of Danbury, Conn., and linear low density polyethylene (25%) commercially designated “16502F3” available from the Montell Corp. of Wilmington, Del. was extruded through a conventional coat hanger die at a die temperature of 246 degrees C. and onto the anchor portions of the corrugated nonwoven just prior to the nip between the second corrugating roll and a cooling roll in an amount appropriate to form a thermoplastic backing layer having a basis weight of 25 grams per square meter with the anchor portions of the formed sheet of fibers embedded in the backing layer.

### Example 2

To demonstrate the use of other patterned corrugating rolls, a web of wipe material was prepared similar to the web in Example 1 except the first corrugating roll was machined such that the entire periphery of the roll was covered with cylindrical protuberances having a diameter of about 4 mm and a center to center spacing of about 5.6 mm. The second corrugating roll was machined such that the entire periphery of the roll was covered with circular depressions having a diameter of about 5 mm. The circular depressions had a center to center spacing on the patterned roll of about 4.6 mm resulting in about 25 percent flat land area between the depressions. The first and second corrugating rolls were mounted such that the protuberances of the first roll meshed with the depressions of the second roll. The same extrudate was used as in Example 1.

### Example 3

A web of wipe material was prepared as in Example 1 above except nonwoven web C2 was used to form a corrugated nonwoven structure.

### Example 4

A web of wipe material according to the present invention was prepared as in Example 1 above except nonwoven web C3 was used to form a corrugated structure. The intermeshing corrugating rolls were machined with axially parallel ridges spaced such that there were approximately 3 ridges per centimeter with a groove between each ridge. Extruded filaments were used to bond to the corrugated nonwoven instead of a continuous extruded film. The same polymer blend as used in Example 1 was used to extrude about 9.4 filaments per centimeter through 0.52 mm orifices at a basis weight of about 55 grams per square meter of web. The filaments were extruded onto the anchor portions of the corrugated nonwoven just prior to the nip between the second corrugating roll and the cooling roll.

### Example 5

A web of wipe material according to the present invention was prepared as in Example 4 above except nonwoven web C4 was used to form the corrugated nonwoven web. An extrudate consisting of about 9.4 filaments per centimeter through 0.52 mm orifices at a basis weight of about 25 grams per square meter of filaments was used to bond the nonwoven.

### Example 6

A web of wipe material according to the present invention was prepared as in Example 2 above except nonwoven web C5 was used to form the corrugated nonwoven web. An extrudate consisting of about 9.4 filaments per centimeter through 0.52 mm orifices at a basis weight of about 25 grams per square meter of filaments was used to bond the nonwoven.

### Example 7

A web of wipe material according to the present invention was prepared as in Example 1 above except a carded nonwoven web consisting of a 35 gram per square meter blend of 3 denier T-224 polyester fibers (80%) from KoSa (Houston, Tex.) and 1.5 denier rayon fibers (20%) from Lenzing Fibres Corp. (Charlotte, N.C.), was used to form the corrugated nonwoven.

### Example 8

A web of wipe material according to the present invention was prepared as in Example 4 above except a carded nonwoven web consisting of 18 denier F1234 polypropylene fibers at a 50 gram per square meter basis weight from Steen & Co. (Schwarzenbek, Germany), was used to form the corrugated nonwoven. The filaments were extruded at a 50 gram per square meter basis weight. The intermeshing corrugating rolls were machined with axially parallel ridges spaced such that there were approximately 4 ridges per centimeter.

### Example 9

A web of wipe material according to the present invention was prepared as in Example 8 above except a carded nonwoven web consisting of 9 denier T196 polypropylene fibers at a 60 gram per square meter basis weight from Hercules Fibers (Bala Cynwyd, Pa.), was used to form the corrugated nonwoven.

### Example 10

A web of wipe material according to the present invention was prepared as in Example 8 above except a carded nonwoven web consisting of 6 denier J01 polypropylene fibers at a 50 gram per square meter basis weight from BP Amoco, was used to form the corrugated nonwoven.

### Example 11

A web of wipe material according to the present invention was prepared as in Example 4 above except a carded nonwoven web consisting of 3 denier Dacron polyester fibers at a 35 gram per square meter basis weight from DuPont (Wilmington, Del.), was used to form the corrugated nonwoven. The filaments were extruded at a 20 gram per square meter basis weight. The intermeshing corrugating rolls were machined with axially parallel ridges spaced such that there were approximately 3 ridges per centimeter.

### Example 12

A web of wipe material according to the present invention was prepared as in Example 11 above except a microporous polypropylene film was laminated to the corrugated nonwoven by introducing the film into the nip formed by the second corrugating roll and the smooth cooling roll. The microporous film was prepared similar to that described in PCT application WO 99/29220 Example 1, having the following composition: 5D45 polypropylene (64 percent, Union Carbide Co.), mineral oil (35%, white oil #31, Amoco Oil & Chemical Co.), and #7 green copper phthalocyanine pigment (1.0%, CI #74260, Sun Chemical Co.). The microporous film had a thickness of 37 microns and a void content of 30%. The microporous film side of this web can be used for removing oil and the other side can be used as a wipe, with or without application of active agents or cleansers.

### Example 13

A web of wipe material according to the present invention was prepared as in Example 12 above except the C4 polypropylene nonwoven web was laminated to the corrugated nonwoven in place of the microporous film.

### Example 14

A web of wipe material according to the present invention was prepared as in Example 1 above except a carded nonwoven web consisting of 3 denier T256 bicomponent fibers at a 35 gram per square meter basis weight from Kosa, was used to form the corrugated nonwoven.

### Example 15

A web of wipe material according to the present invention was prepared as in Example 1 above except a carded nonwoven web consisting of 3 denier T224 polyester fibers at a 35 gram per square meter basis weight from Kosa, was used to form the corrugated nonwoven.

### Example 16

A web of wipe material according to the present invention was prepared as in Example 1 above except a carded nonwoven web consisting of a 35 gram per square meter blend of 3 denier T-256 bicomponent fibers (90 percent) from KoSa and HiQ cotton fibers (10%) from Barnhardt Manufacturing (Charlotte, N.C.), was used to form the corrugated nonwoven.

### Example 17

A web of wipe material according to the present invention was prepared as in Example 8 above except a carded nonwoven web consisting of 3 denier Fiber Visions polypropylene fibers at a 35 gram per square meter basis weight from HyComfort ( ), was used to form the corrugated nonwoven. The filaments were extruded at a 20 gram per square meter basis weight. The nonwoven web described as C1 above was laminated to the corrugated nonwoven by introducing the nonwoven into the nip formed by the second corrugating roll and the smooth cooling roll.

### Example 18

A web of wipe material according to the present invention was prepared as in Example 17 above except a carded nonwoven web consisting of 3 denier T224 polyester fibers at a 35 gram per square meter basis weight from Kosa, was used to form the corrugated nonwoven.

### Example 19

A web of wipe material according to the present invention was prepared as in Example 8 above except a carded nonwoven web consisting of 9 denier T-196 fibers at a 55 gram per square meter basis weight from FiberVisions ( ), was used to form the corrugated nonwoven. The filaments were extruded at a 40 gram per square meter basis weight. A 60 gram per square meter spunlaced nonwoven web, 50 percent rayon and 50 percent polyester, available from PGI Nonwovens of Charleston, S.C. was laminated to the corrugated nonwoven by introducing the nonwoven into the nip formed by the second corrugating roll and the smooth cooling roll. To demonstrate the use of the webs of the present invention as a cleansing wipe, the corrugated side of the above web was coated with a cleansing formulation consisting of 20 percent Cocamidopropyl betaine available from Henkel as Velvetex BA-35, 79.2 percent water, and 0.8 percent Hydroxypropyl methyl cellulose available as Methocel from Dow. The coated web was then dried in an oven at 66° C. resulting in a 15 gram per square meter dry coating. The resulting web can be used as a cleansing wipe for hair and skin by wetting with water. The corrugated side of the wipe can be used for skin exfoliation.

The absorbent capacity results below show that the wipes of the present invention have a significantly higher absorption capacity compared to corresponding flat non-corrugated webs and commercially available cosmetic applicators. The wipes of the invention generally have a water or oil absorption capacity of at least 20 percent greater than a corresponding flat wipe of the same nonwoven material, preferably at least 40 percent greater, particularly with harder to release oil based active agents, where the percent efficiency is significantly higher (e.g., at least 5 percent higher). Additionally, the webs more readily release the active agent in response to applied pressure. The results demonstrate that a corrugated web has a structure that is highly suitable for absorbing or carrying and delivering various active agents.

TABLE 1

| | Water | Water | | Oil | Oil | |
|---|---|---|---|---|---|---|
| Mate- | Absorbent | Deliv- | % Water | Absorbent | Deliv- | % Oil |
| rial | Capacity | ery | Efficiency | Capacity | ery | Efficiency |
| C1 | 6.5 | 5.8 | 89 | 7.7 | 5.3 | 69 |
| 1 | 9.2 | 8.1 | 89 | 12.3 | 9.3 | 75 |
| 2 | 10.1 | 9.1 | 90 | 10.7 | 8.0 | 75 |
| C2 | | | | 6.8 | 3.4 | 50 |
| 3 | | | | 8.4 | 5.4 | 64 |
| C3 | 3.9 | 3.9 | 99 | 10.6 | 8.9 | 83 |
| 4 | 11.7 | 11.7 | 99 | 21.6 | 19.8 | 91 |
| C4 | 3.2 | 3.1 | 99 | 9.3 | 7.8 | 85 |
| 5 | 8.3 | 8.2 | 99 | 17.5 | 16.3 | 93 |
| C5 | 1.8 | 1.7 | 99 | 5.9 | 4.7 | 80 |
| 6 | 3.8 | 3.7 | 99 | 10.8 | 10.0 | 92 |
| 7 | 23.8 | 23.4 | 98 | 33.5 | 29.4 | 88 |
| C6 | 14.0 | 9.2 | 66 | 13.1 | 9.5 | 72 |
| C7 | 13.3 | 8.6 | 64 | 29.5 | 21.2 | 72 |
| 8 | 5.8 | 5.6 | 96 | 12.7 | 12.0 | 95 |
| 9 | 5.9 | 5.7 | 97 | 10.9 | 10.3 | 95 |
| 10 | 8.8 | 8.6 | 98 | 13.7 | 13.1 | 96 |
| 11 | 14.2 | 14.0 | 99 | 25.2 | 23.9 | 95 |
| 12 | 11.9 | 11.7 | 98 | 16.5 | 15.1 | 92 |
| 13 | 16.8 | 16.4 | 98 | 19.3 | 17.5 | 90 |
| 14 | 14.3 | 14.2 | 99 | 19.9 | 18.9 | 95 |
| 15 | 20.4 | 20.1 | 99 | 22.3 | 20.6 | 92 |
| 16 | 20.2 | 20.0 | 99 | 24.4 | 22.1 | 90 |
| 17 | 10.8 | 9.6 | 88 | 14.2 | 12.3 | 86 |
| 18 | 13.1 | 11.8 | 90 | 15.6 | 13.4 | 86 |

Table 2 below shows the increased ability of the corrugated webs of the invention to form greater foam heights than the corresponding comparative flat webs. The foam heights are generally at least 15 percent higher than the corresponding flat webs, preferably at least 30 percent higher.

TABLE 2

| Material | Foam Height |
|---|---|
| C2 | 6.0 |
| 3 | 7.8 |
| C3 | 4.8 |
| 4 | 8.5 |
| C4 | 6.0 |
| 5 | 6.9 |
| CS | 4.6 |
| 6 | 8.6 |

We claim:

1. A package containing at least one soft fibrous wipe laminate; the wipe laminate comprising:
a nonwoven wipe layer formed from a nonwoven web comprised of fibers formed from thermoplastic polymers, copolymers or blends; and
a backing layer bonded to the wipe layer where the nonwoven fibrous web has bonded regions and unbonded regions, the unbonded region forming arcuate mounds.

2. The package of wipes of claim 1 wherein the wipe layer is a nonwoven fibrous web having a basis weight of from 10 to 100 grams/m² and the fibrous wipe layer has a z-direction from the backing of at least 0.5 mm, and the arcuate mound comprises 20 to 99 percent of wipe laminate.

3. The package of wipes of claim 2 wherein the wipe layer has an impregnated active agent.

4. The package of wipes of claim 2 wherein the nonwoven wipe layer is formed of at least 10 percent bondable thermoplastic fibers by weight.

5. The package of wipes of claim 2 wherein the nonwoven wipe layer is formed of at least 20 percent bondable thermoplastic fibers by weight.

6. The package of wipes of claim 4 wherein the nonwoven wipe layer contains at least in part hydrophilic fibers.

7. The package of wipes of claim 6 wherein hydrophilic fibers are natural fibers.

8. The package of wipes of claim 4 wherein the backing layer is a thermoplastic layer that is autogeneously bonded to the nonwoven wipe layer.

9. The package of wipes of claim 8 wherein the backing layer is a thermoplastic film.

10. The package of wipes of claim 8 wherein the backing layer is a plurality of mutually parallel thermoplastic filaments.

11. The package of wipes of claim 2 wherein the backing layer has a basis weight of from 15 to 150 g/m².

12. The package of wipes of claim 11 wherein the nonwoven wipe layer has a basis weight of from 15 to 50 g/m².

13. The package of wipes of claim 2 wherein the fibers forming the wipe layer are predominantly 1 to 50 μm in diameter.

14. The package of wipes of claim 2 wherein the wipe material laminate is from 10 to 100 cm² and has a basis weight of from 30 to 300 g/m².

15. The package of wipes of claim 2 wherein the wipe material laminate is from 20 to 50 cm² and has a basis weight of from 40 to 100 g/m².

16. The package of wipes of claim 2 wherein the package of wipe laminates contain at least 10 wipes.

17. The package of wipes of claim 2 wherein the arcuate mounds are regular in shape.

18. The package of wipes of claim 2 wherein the mounds are irregular in shape.

19. The package of wipes of claim 2 wherein the arcuate mounds comprise from 50 to 95 percent of the cross sectional area of the wipe laminate.

20. The package of wipes of claim 2 wherein 99.5 to 50 percent of the cross sectional area of the nonwoven fibrous wipe layer in the wipe laminate is unbonded.

21. The package of wipes of claim 2 wherein the bonded portion of the nonwoven wipe layer is are bonded to the backing layer, have a width of from 0.01 to 0.2 cm and adjacent bonded portions are spaced on average from 0.1 to 2.0 cm apart.

22. The package of wipes of claim 2 wherein the z-direction loft is from 1 to 5 mm.

23. The package of wipes of claim 2 wherein the nonwoven fibrous wipe layer comprises at least 50 percent thermoplastic fiber.

24. The package of wipes of claim 2 wherein the nonwoven fibrous wipe layer comprises at least 75 percent thermoplastic fiber.

25. The package of wipes of claim 2 wherein the wipes have at least 20 percent greater water or oil absorption capacity than a corresponding nonwoven wipe layer without arcuate mounts.

26. The package of wipes of claim 2 wherein the wipes have at least 40 percent greater water or oil absorption capacity than a corresponding nonwoven wipe layer without arcuate mounts.

27. The package of wipes of claim 2 wherein the wipe forms foam height at least 45 percent higher than a corresponding nonwoven wipe layer without arcuate mounds.

28. The package of wipes of claim 2 wherein the wipe forms foam height at least 30 percent higher than a corresponding nonwoven wipe layer without arcuate mounds.

29. The package of wipes of claim 11 wherein nonwoven wipe layer is a carded fiber nonwoven web.

30. The package of wipes of claim 2 wherein the fibers forming the nonwoven wipe layer of from 1 to 70 μm in diameter.

31. The package of wipes of claim 30 wherein the fibers forming the nonwoven wipe layer of from 40 to 70 μm in diameter.

32. The package of wipes of claim 30 wherein the fibers forming the nonwoven wipe layer of from 1 to 30 μm in diameter.

33. The package of wipes of claim 32 wherein the fibers are a blend including compression resistant fibers.

34. The package of wipes of claim 2 wherein the nonwoven fibrous web is formed of at least 10 percent hydrophilic fibers.

35. The package of wipes of claim 2 wherein the nonwoven fibrous web is formed of at least 10 percent fibers having a denier of 9 or greater.

36. The package of wipes of claim 2 wherein the backing is formed of at least 10 percent absorbent fibers.

37 The package of wipes of claim 2 wherein the backing is oil absorbent.

38. The package of wipes of claim 2 wherein the backing is preimpregnated with active agent.

39. The package of wipes of claim 3 wherein the wipe layer is wet.
